(19) United States
(12) Patent Application Publication
(10) Pub. No.: US 2021/0358068 A1
(43) Pub. Date: Nov. 18, 2021

(54) METHOD FOR ISSUING A VERIFIED HEALTH PASS, USE THEREOF FOR ENTERING A VENUE AND CONTACT TRACING METHOD

(71) Applicant: BBL HEALTHCARE SOLUTIONS LTD, High Wycombe (GB)

(72) Inventors: Bronek BOSZCZYK, High Wycombe (GB); Robert Anthony LEE, High Wycombe (GB); Nicholas Charles BIRCH, High Wycombe (GB); Robert Darren LEE, High Wycombe (GB)

(73) Assignee: BBL HEALTHCARE SOLUTIONS LTD, High Wycombe (GB)

(21) Appl. No.: 16/874,180

(22) Filed: May 14, 2020

(51) Int. Cl. G06Q 50/26; G16H 10/65; G16H 50/30; G07C 9/23; G07C 9/27; G16H 50/80; G06F 21/60; G06F 21/31; G06K 7/14; A61B 5/1171; H04W 4/029; H04W 4/30

(52) U.S. Cl. CPC G06Q 50/265; G16H 10/65; G16H 50/30; G07C 9/23; G07C 9/27; G16H 50/80; H04W 4/30; G06F 21/31; G06K 7/1417; G06K 7/1413; A61B 5/1176; H04W 4/029; G06F 21/602

(57) ABSTRACT

The provision of a pass that may be used in the field of infectious disease prevention and control and, more specifically, a method for the provision of a verified health pass that is indicative for at least one health status of an individual and can be used for allowing entry to a venue depending on said specific health status. The verified health pass may further be used for linking an individual's identity to an associated proximity detection device to allow contact tracing of individuals inside a venue while minimizing transfer of personal data.

The claimed invention generally relates to the provision of a pass that may be used in the field of infectious disease prevention and control. More specifically, this application relates to a method for the provision of a verified health pass on an individual's mobile device that is indicative for at least one health status of said individual and can be used for allowing entry to a venue if said specific health status meets predefined criteria. The verified health pass may further be used for linking an individual's identity to an associated proximity detection device to allow contact tracing of individuals inside a venue while minimizing transfer of personal data.

Recent years have seen an increasing spread of regional and global diseases, the transmission of which may be facilitated by heightened mobility of the global populace. Ranging from Severe Acute Respiratory Syndrome (SARS) to Middle East Respiratory Syndrome (MERS), from Measles to Avian Influenza and to Ebola and the like, such infectious diseases may cause a great burden to people due to the high transmission rate and the health impact, including the economic loss of work force and even a high rate of fatality.

In some instances, the spread of a disease occurs through close interactions between an infected individual and another individual. This is especially true in an enclosed environment such as an office space, school, public transit, etc. Also, the situation is aggravated by an infected individual visiting public facilities without knowing that he or she is infected. Due to close proximity of individuals in public venues, the disease may spread rapidly.

To prevent the transmission of an infectious disease, it is important to identify individuals of a community that are infected at an early stage, to keep them isolated for the time that the individual is infectious (quarantine).

Before allowing entry of a large numbers of individuals to a venue it would thus be highly beneficial to exclude people whose health status may present a risk to others.

There is a need for determining a health status—for instance an infection status or immunity status—of an individual to determine whether said individual might pose a risk to other individuals and under which circumstances. While allowing verification of an individual's identity, transfer of personal data should be kept at a minimum and meet common data safety standards.

This first object is achieved by the method according to Claim 1 of the present application. Preferred embodiments are subject of the dependent Claims.

The method of the present invention is used for issuing a verified health pass to be stored on an individual's mobile device, e.g. a mobile phone or other personal communication device. To this end, the method comprises the steps of:
verifying an individual's identity;
receiving at least one verified health status of the individual, said verified health status being indicative of a first health parameter and being determined by a health care authority;
transmitting the verified health status and verified identity to a remote server to create a verified health pass, said verified health pass including an optical or digital code containing encrypted information on the identity and health status of the individual; and
transmitting the verified health pass to a mobile device or portable computing device of the individual.

Within the context of this application the term “individual” generally refers to people but does also include other living entities, in particular animals.

The term “disease” includes any known—in particular infectious—disease caused by one of a microorganism, a microbe, a virus, a bacterium, an archaeum, a protozoan, a protist, a fungus or a microscopic plant. The virus can comprise influenza, corona virus or HIV. The bacterium can comprise Mycobacterium tuberculosis. The protozoan can comprise malaria.

In other embodiments, the disease is a chronic disease or condition, such as e.g. diabetes, prediabetes, insulin resistance, metabolic disorder, obesity, or cardiovascular disease.

The verified health pass may be transmitted and stored on a variety of mobile devices, such as an individual's mobile phone, tablet or other communication device. Alternatively, the health pass also be printed on a wearable device, peace of clothing or accessories, or stored in a memory of an implantable device.

To avoid trading of the verified health pass, the inventive method ensures that the identity of an individual is linked to their verified health pass.

As the identification and health data of the individual are stored encrypted in the digital or optical code of the verified health pass, protection of private information is ensured, such that it complies with regulatory requirements. Data protection may be achieved by storing the data in a fully encrypted local cloud based storage database that does not leave the country of origin, such as an Amazon Simple Storage Service (Amazon S3 Web Service).

The verified health pass may also be stored in an application (app) software running on a mobile phone or other personal communication device that preferably includes a display, communication function and memory function. The digital or optical code may be displayed upon opening the app.

In summary, the verified health pass thus provides an identity linked health status that is indicative for a specific health parameter, such as e.g. immunity status, infection status (i.e. presence or absence of an infection) or the like. Preferred uses of this verified health pass will be explained in more detail further below.

The identity verification is preferably performed by a remote ID verification service. In a preferred embodiment it involves video identification or involves uploading of a photo identity of a passport or ID card and a facial photo to the ID verification service. The ID verification service may analyse a person's facial biometric information in a selfie or video, and match this to a government issued ID. One preferred example of video identification is the service of WEBID SOLUTIONS GMBH. The method they developed for remotely identifying a person's identity is disclosed e.g. in U.S. Pat. No. 9,911,035 B1.

The verified identity of the individual can be transmitted as a non-identifiable code which can only be connected to the individual with the necessary decoding information. This ensures secure handling of any identifiable data.

In line with the present invention, the verified identity is then associated with a verified health status. Preferably, the verified health status includes information of at least one of i) immunity status, ii) infection status, iii) inflammation status and iv) vaccination history.

In case of the verified health status being indicative of an infection status for a specific virus, said infection status is preferably determined by a test performed by a health care professional. The test may involve a virus test, which is a test aimed to identify viruses or virus-specific infection markers in specimens such as blood, serum, sputum, urine, cerebrospinal fluid or other possible infected tissue. More preferably, the infection status is determined with the aid of an antigen test and/or an antibody test. An antigen test is a diagnostic test suitable for directly detecting the presence or absence of an antigen to see if there is any active infection going on, with the virus still present in the individual. Common examples of antigen tests include strep tests for streptococcal antigens, influenza tests for influenza virus antigens and malaria antigen detection tests for Plasmodium antigens. An antibody test will check whether the individual has had a previous encounter with the virus and their immune system responded accordingly by producing antibodies. One inherent advantage of an antigen test over an antibody test (such as antibody-detecting HIV tests) is that it can take time for the immune system to develop antibodies after infection begins, but the foreign antigen is present and can therefore be detected right away.

The verified health pass may indicate that an individual is “safe” with respect to a specific virus in two cases: either if the antibody test is positive (competent immune response) and the antigen test is negative (no longer carrying the virus), or if both of the antibody test and antigen test are negative—which indicates that the individual has not yet encountered the virus.

The verified health status may also be indicative of an individual's immunity to a certain disease. The development of immunity to a pathogen through natural infection is a multi-step process that typically takes place over 1-2 weeks. In case of a viral infection, the body's first reaction is a non-specific response, that is followed by an adaptive response. The latter involves the production of antibodies that specifically bind to the virus and the production of T-cells that recognize and eliminate other cells infected with the virus. This combined adaptive response may leave a lasting memory of the infection and prevent re-infection by the same virus, at least for a certain time period. The presence of certain antibodies against a specific virus in the blood is recognized as one indication that an individual could be immune to subsequent infection by this virus. The antibodies can either be produced naturally by recovering from the disease, or triggered through vaccination.

For that reason, if the health status includes information on an immunity status, an individual's immunity may be determined based on the presence or absence of antibodies, T-cells, memory B cells, intracellular or cell surface proteins or glycoproteins, or cytokines indicative for a specific disease in a tissue sample (generally blood or serum) of the individual.

Immunities—whether attained by past infection or immunization—have different durations. Some immunities are life-long, e.g. after a measles vaccination. Other immunities may be short-lived, such that individuals having recovered from an infection may be re-infected by the same virus after e.g. a year. For that reason, the verified health pass is preferably programmed such that it expires after a predefined time limit with respect to a certain verified health status. For instance, an immunity status may expire after a presumed duration of immunity, thereby prompting the individual to be retested at regularly intervals.

The health status indicative for a certain health parameter may also change in other events, for instance if an individual has recently come into close contact with an other individual that is diagnosed with an infective disease.

In line with the present invention, the verified health pass includes an optical or digital code containing encrypted information on the identity and at least one health status of the individual. The code may be in form of a QR code, a barcode or digital code which can be scanned by a scanning device or transmitted via Bluetooth, wireless or related technology to a receiving device.

The method as claimed in any of Claims 1 to 5, wherein the optical code is a QR code and preferably only contains encrypted information on the verified health status and identity of the individual. The QR code may be scanned by an official to check the individual's health status.

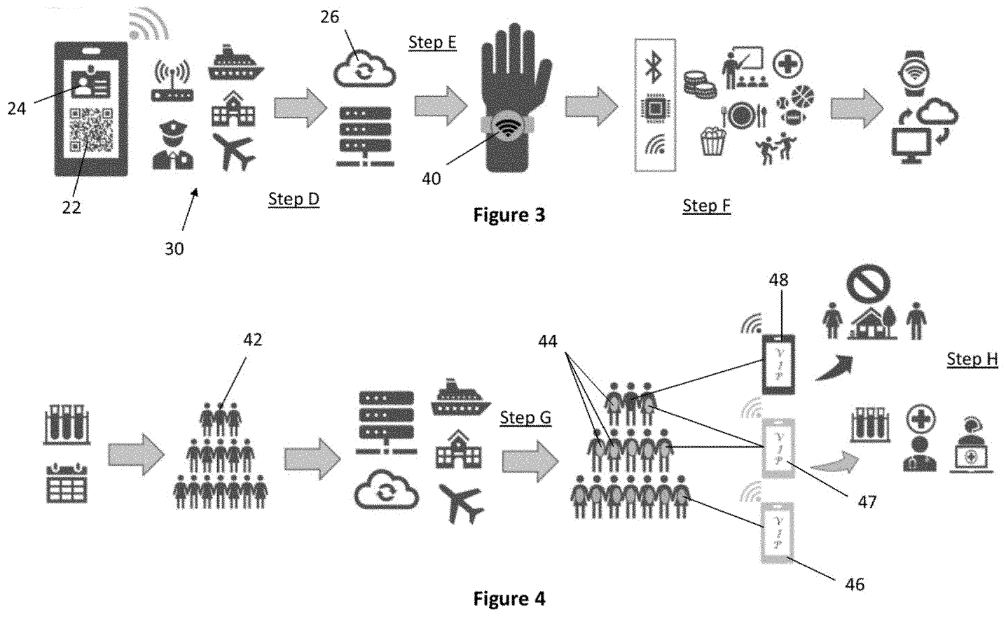

As will be described in more detail in connection with the description of FIG. 3 further below, in a preferred embodiment, the digital or optical code on the verified health pass is used for allowing entry to a venue. More specifically, the optical code allows entry to a venue, if a specific verified health status meets at least one predefined criterion, said predefined criterion preferably including at least one of
i) absence of infection,
ii) presence of immunity to a specific disease,
iii) absence of recent intercontinental travel history,
iv) absence of recent travel history into countries presenting a health risk,
v) lasting vaccination for a specific disease, and
vi) absence of a specific health issue (such as hypertension, diabetes, cancer, immune deficiency, obesity).

For instance, an individual with a recent travel history in a country associated with a particular disease, said individual may be required to provide evidence of immunity against this specific disease, as a condition of entry.

The verified health pass can thus be used in epidemic or pandemic situations to allow or deny entry of individuals into defined areas (“venues”) based on their associated health status.

Issuing a specific health status could include risk stratification based on predictive analytics. For example, if a health status indicates severe asthma that individual might be denied entry into nightclub but allowed to enter casino where lesser density of people is maintained. The verified health pass can also be used to selectively allow entry to certain rooms based on this risk strategy. It is further possible that the verified health pass can be adapted via remote data push to modify a certain status—e.g. change from green to yellow as a warning if for example the density becomes too high.

In addition of being indicative of a certain health status, inter alia an infection or immunity status, the verified health pass may also provide information on allergies, health issues like diabetes or hypertension, drug consumption, sexually transmitted diseases or doping tests. For instance, in the adult film industry, performers are required to be regularly tested for sexually transmitted disease (STD). The verified health pass would provide an ID verified STD status for entry to the set. The same principle could be used for dating apps where for example HIV and HEPc (Hepatitis C) status are included and cumulatively displayed as a “green pass” so that dating participants know their partner has a certain status. This could also be used for information in adult venues to restrict certain services for those with an infective status (e.g. HIV or Hepatitis). This would greatly improve the safety of clients and workers in the sex industry.

In this respect, the verified health pass may also take into account the results of a risk stratification performed using predictive algorithms. Predictive analytics can be applied that use health care and personal data from the patient and combine this data with e.g. the risk of reinfection according to the infection statistics obtained through collective use. Over time this allows to predict if personal parameters and health care conditions predispose an individual to a greater risk of infection or reinfection than others (e.g. if the person is over 60 and has diabetes the risk of acquiring an infection is higher than in a healthy 18 year old). Artificial Intelligence (AI) may also be employed to allow automated learning in the field of predictive analytics.

Data from a risk stratification through predictive algorithms could further be used to provide a risk stratified pass allowing access to certain types of venues. The verified health pass may then be generated with different security of risk levels, e.g. such that entry to a venue may be granted or denied depending on the number of individuals present in the venue. For example, an elderly person with diabetes may be at a low risk for attending a classical music concert with a seating arrangement but would be at high risk in a highly density environment such as a night club. Depending on the risk level, entry to a specific venue might only be allowed if the individual agrees on certain security measures, such as wearing a minimum of personal protective equipment (e.g. face mask).

Artificial Intelligence (AI), such as Natural Language Processing or machine and deep learning techniques, may be used to aid collecting medical information and store it, e.g. in a special app that contains the verified health pass. Such medical information may include common health parameters used to indicate whether an individual has diabetes, is obese, suffers from a heart condition, has asthma, has COPD, and/or is taking prescribed medication and in which quantity. Such information may be collected in digital text files that may contain a range of clinical terminology identifiable by different systems such as SNOMED, for instance. The collected data may then be evaluated using AI to identify clinical correlations and to develop predictive algorithms that predict the trajectory of any particular individual including the risk classification of each type of individual tested. Advice and guidance that is specific for a particular individual can therefore be offered.

Collection of data—in particular medical or other personal information—is preferably done in an anonymised manner. To this end, differential privacy methodology may be employed. In simple terms, random «noise» may be inserted to the data file of a specific individual to thereby falsify the data in the individual format whilst retaining the accuracy in the aggregated form.

In practice, the verified health pass might also be used for restricting access or enabling access to high-risk individuals, such as individuals with immune deficiencies (e.g. in a cancer ward where patients are at risk of any type of viral infection). Access might in this case be dependent on the presence or absence of recent infections and/or vaccinations.

The use of a verified health pass as described above therefore presents a potent measure for allowing individuals to socialize with one another while reducing the risk of negatively affecting the individual's health status, e.g. by transmitting an infectious disease.

Nevertheless, there is always a certain risk of error in the determination of the health status, e.g. if an individual is incorrectly identified as immune, and also the risk of “conversion”, meaning that an individual having a health status indicating the absence of an infection later showing symptoms and being diagnosed with an infectious disease.

For that reason, another key measure in infection control is tracing anyone who may have come into contact with an infected individual (“contacts”) for purposes of screening, diagnosis, and limiting further transmission of infectious agents. This process is generally referred to as “contact tracing” or “contact tracking”. Contact tracing has been used in the management of outbreaks of highly infectious and dangerous diseases, such as measles, sexually transmitted infections (including HIV), blood-borne infections, some serious bacterial infections, and novel infections (e.g. SARS-CoV and SARS-CoV-2). Partner notification, also called partner care, is a subset of contact tracing aimed specifically at informing sexual partners of an infected person for managing sexually transmitted infections (STIs).

It has been proposed to use proximity-based protocols for contact tracing.

For instance, US-A1-2015/0100330 discloses a method and system for detecting and identifying infectious and hazardous sites based on receiving location data from a mobile device associated with a user and receiving health data for the user.

U.S. Pat. No. 7,993,266 B1 discloses a personal apparatus that transmits proximity data to a computer controller and to each other where the computer controller transmits a notification to the personal apparatus of at least one human.

US-A1-2013/0275160 discloses a method that involves receiving location data for a mobile device and comparing a mobility pattern derived from the received location data with a mobility pattern of first users to determine occurrence of a proximity event.

Challenges with contact tracing may arise related to issues of privacy and confidentiality.

There is thus also a need for contact tracing methods that allows contact tracing within areas where social distancing is not possible. While allowing identification of individuals having been in close contact to an infected individual, the method should also ensure privacy and confidentiality of the collected data.

This second object is achieved by the method according to Claim 9 of the present application. Preferred embodiments are subject of the dependent Claims.

In general terms, the present invention provides a method that allows tracking of movement of individuals in close proximity. In one embodiment, the present invention relates to a method for the provision of a verified health pass on an individual's mobile device that is indicative for at least one health status of said individual and can be used for linking an individual's identity to an associated proximity detection device to allow contact tracing of individuals inside a venue while minimizing transfer of personal data.

More specifically, a method for contact tracing in a venue is proposed, said method comprising the steps of:
providing a plurality of proximity detection devices each of which including a proximity sensor and a battery, and being configured to enable reception, storage and transmission or read-out of data;
for each individual entering a venue, scanning or receiving a code, verifying the individual's identity and generating a unique identification code linked to the individual's identity;
associating one of the proximity-detection devices to each individual and transmitting the individual's unique identification code to the associated proximity-detection device;
with the aid of the proximity sensor detecting and recording interactions between two individuals if an interaction is within the criterion thresholds of least one predefined criterion selected from the group of duration, proximity, and frequency of the interaction; and
transmitting the information on recorded interactions to a secure server database.

This method allows tracing of anyone within a specific area who has come in contact with an individual carrying a disease and whether the contact was at such a proximity and a duration that a transmission of the disease might have occurred.

Specifically, a first proximity detection device of an individual detects a second proximity detection device of another individual, e.g. via low-energy radio frequency communication. The first device records contact data, including an identification of the second device, if the contact between the first proximity detection device and a second proximity detection device is within the criterion thresholds, e.g. if the devices are within the specified distance from one another, and optionally for a specified duration. This recorded interaction is then stored in a secure server database. The data on recorded interactions may be first stored in a storage of the proximity detection devices and later transmitted to the secure server database or the recorded interactions may be instantly transmitted. The recorded data is preferably stored encrypted.

The proximity detection device is preferably waterproof and can be worn in any environment, including in rainy environments or environments with high humidity, such as changing rooms, beach, saunas.

The proximity detection device generally includes a central processing unit (CPU); a RF communication unit including a sender and a receiver, a battery as well as a housing. The CPU generally allows for the interpretation and execution of system commands, and provides the necessary temporary memory space for storing intermediate data. The system commands preferably include one or more of the following: software, user control, real-time signal processing algorithms for signal detection, analysis and reporting, as well as output generation.

The proximity detection device generally further includes a memory. Even though a memory is not required in every case, in particular if contact data is continuously transferred to the secure server database, it is preferred that there is at least some storage capacity within the proximity detection device to allow temporary storage of data—e.g. in particular recorded contact data in case of a problem hindering immediate data transfer to the server database.

The secure server database may be located in a cloud environment. This permits instant transmission to and storage of recorded interactions in the cloud, thereby reducing the need of storage space on the proximity detection device.

The contact tracing method therefore presents a potent tool to help reduce spreading of contagious diseases, such as tuberculosis, haemorrhagic fever, chicken pox, avian flu, Severe Acute Respiratory syndrome (SARs), Coronavirus (COVID), etc. It not only reduces the likelihood of individuals coming into contact with another individual carrying a disease, but in case that an individual is later diagnosed with a disease, the method also allows that anyone who came into contact with the infected individual can be identified.

Preferably, the optical or digital code is contained in a verified health pass as presented above. Preferably the verified health pass is stored in a specific app on a mobile phone or other communication device that also includes identification data of the individual associated with the verified health pass.

For gaining entry into the venue, the digital or optical code on the verified health pass is scanned or otherwise read out and the unique identification code generated thereby is then preferably linked to the verified health pass of the individual entering the venue.

Thus, whether access to the venue is granted or not depends on the information contained in the verified health pass. For instance, access to the venue may only be granted if the or a particular health status meets predefined criteria, such as absence of an infection and/or immunity to a certain disease.

In a preferred embodiment the tracking occurs in a venue equipped with—preferably high resolution—Real Time Locating System (RTLS). RTLS is a form of local positioning system that is generally used in indoor and/or confined areas, such as buildings, and is usually independent of GPS or mobile phone tracking. RTLS technology is generally uses some form of radio frequency (RF) communication, but some systems use optical (usually infrared) or acoustic (usually ultrasound) technology instead of or in addition to RF. RTLS tags are affixed to mobile items to be tracked or managed. RTLS reference points, which can be either transmitters or receivers, are spaced throughout a building (or similar area of interest) to provide the desired tag coverage.

Alternatively, the proximity sensor may use other communication technologies, such as Radio Frequency Identification (RFID), and Near Field Communication (NFC).

As mentioned, an interaction between two individuals is recorded if it is within the criterion thresholds of least one predefined criterion selected from the group of duration, proximity, and frequency of the interaction.

Since many contagious diseases are transmitted through the inhalation of airborne droplets, close contact between individuals is a preferred criterion for recordal of an interaction. For instance, an interaction is recorded if two proximity detection devices come into contact at a distance of less than 1.5 m, preferably less than 2 m.

The predefined criterion for recording an interaction is preferably adjustable by a program stored in the proximity detection device. This allows adjusting the criterion depending on the type and transmission route of a disease.

The proximity detection device is preferably sized and configured to be worn around a body part of the individual. It may be configured e.g. as a tag, necklace, bracelet, ring or ankle band. In a specifically preferred embodiment, the proximity detection device is a bracelet.

If configured as a wearable device or item, each proximity detection device is preferably securely attached to the associated individual upon entering a venue, such that casual removal is not possible. In one embodiment, the proximity detection device is configured as a bracelet having a safety lock or safety seal, which cannot accidently or inadvertently be released. Safety lock for wearables, e.g. jewellery, are known in the art. Non limiting examples are disclosed e.g. in U.S. Pat. No. 5,870,803, WO-A1-1998036656, or U.S. Pat. No. 9,730,497B2. The proximity detection device may also be configured as a wearable device that requires the use of a remover, e.g. provided by an authorized individual such as a staff member of the venue, to remove the proximity detection device without damage.

Alternatively, the proximity detection device may also be configured as an implant for subcutaneous implantation. In this embodiment, the proximity detection device can be used for contact tracing also outside a specific venue.

In a preferred embodiment, upon scanning the optical or digital code at the venue entry, information on the time, date and location are transmitted to the secure server database. This facilitates later extraction and analysis of the recorded data.

In a preferred embodiment, in case of an individual being diagnosed with an infectious disease, the method further includes the steps of:
accessing the database and based on the recorded interactions by the proximity detection device associated to the individual carrying the disease, and
determining potentially infected individuals.

The contact tracing method of the present invention therefore preferably allows the recorded interaction data to be analyzed for any contact and duration (which can be modified according to current health care guidelines) that occurred between two individuals within the venue.

In the above embodiment, the contact tracing method of the present invention therefore presents a potent tool to help reduce spreading of contagious diseases, such as tuberculosis, haemorrhagic fever, chicken pox, avian flu, Severe Acute Respiratory syndrome (SARs), Coronavirus, etc. It not only reduces the likelihood of individuals coming into contact with another individual carrying a disease, but in case that an individual having entered a venue and later being diagnosed with a disease, the method also allows to trace and identify anyone who came into contact with the infected individual within said venue.

The potentially infected individuals are preferably informed, preferably by sending a notification to their personal mobile devices, that their health status might have been compromised. Where required, a change of the verified health pass, in particular if it is caused by a change in infection status, such information may also be instantaneously transferred to health care authorities.

If entry to a venue occurred with the aid of scanning of a code of a verified health pass as described within this application, it is particularly preferred that potentially infected individuals are notified and their verified health status on their mobile device is changed. For instance, the verified health pass may show a verified health status that is no longer “safe”, i.e. absent of a specific disease, but is shown as “potentially compromised”, indicating a potential infection. The notified individuals are preferably further prompted to get retested and potentially isolated until the infective status is confirmed.

The verified health pass may also be provided with a warning function. A warning may be issued in the form of sound, colour change and/or vibration of the mobile device on which the verified health pass is stored. If the verified health pass is incorporated into an application software running on a mobile phone or other communication device, the warning can be issued via said communication device.

In one embodiment, predictive analytics could be applied in real time to warn users if a critical threshold is passed—e.g. a density of visitors or presence of individuals with a defined health care condition placing others at risk. A warning could also be issued if an individual with an active infection of a specific disease comes into proximity of persons not vaccinated against this (or related scenario of infectious diseases) another example as a person not vaccinated for flu entering a hospital.

The verified health pass may also be linked to or stored on a personal tracking device that is not exclusively worn inside a specific venue but in daily life. Such tracking devices are known and are used to measure various health parameters including temperature-related symptoms (e.g. fever, hypothermia) using an infrared video device, audible symptoms (e.g. coughing, sneezing, sniffling) using an audio monitoring device, heart frequency, blood sugar levels, or other bodily or substance parameters that are or become detectable. If a certain health parameter or combination of health parameters are found to be abnormal, e.g. above or below a certain threshold level, this may trigger issuance of a warning sign or sound and may be accompanied with a change in a verified health status. For example, the combination of an elevated pulse rate and body temperature in absence of adequate physical motion/exertion (measured as movement) could indicate infection and trigger the tracking device emitting a visual sound or sense warning. If the condition persists for a predefined time period, this may induce a change of the infection status indicated on the individual's associated verified health pass.

If the venue is a high risk area, a change in an individual's associated verified health pass, e.g. due to the individual's infection status having changed from “safe” to “infected”, might also have the effect that a warning notification is sent to staff members of the venue, such that they can take appropriate action for identifying, real time tracking (with the aid of the proximity detection device), treating and quarantine potentially infected individuals, as necessary. The proximity detection device may then be used for identifying all individuals having come into contact with this infected individual inside the venue. Additional measures, such as denying exit of these individuals from the venue may be taken to allow immediate and effective quarantine.

To ensure data security, differential privacy methodology may be employed. In one embodiment, random «noise» is inserted to an individual's data file thereby falsifying the data in the individual format whilst retaining the accuracy in the aggregated form. Also, pseudonymisation techniques may be applied where specific individuals need to be identified following detection of infection. Another option is the use of an approach taken by AI/NLP specialist company SAVANAMED (MEDSAVANA) that has developed a system known as «Natural Privacy» that leverages the mistakes naturally embedded in NLP (copy paste, spelling errors, false acronyms etc.) and thereby falsifies the data at the individual level whilst retaining the accuracy in aggregated format. This could allow using the data without explicit consent from each individual for the benefit of the wider public interest.

The present invention will be further described in detail in connection with the attached figures, which show an exemplary process of a verified health pass being issued and being indicative of an infection status with COVID-19.

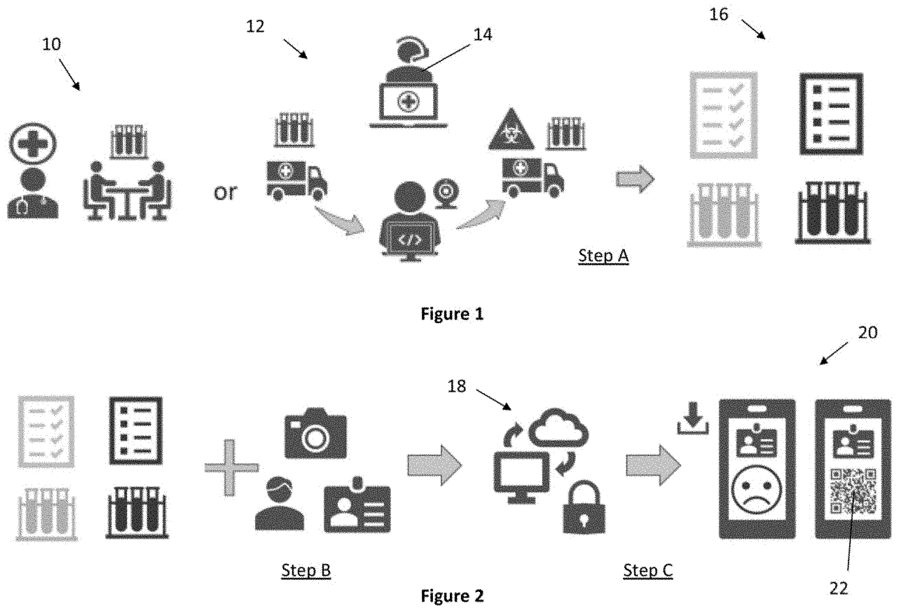

FIG. 1 shows a schematic representation of a testing step (Step A) in which an individual is tested for infection with a certain disease.

FIG. 2 shows a schematic representation of an identification step (Step B) in which the identification of an individual is verified and the verified identity of said individual is linked to a verified health status indicative of the results from testing step (Step A). Both information on the verified identity and the verified health status of the individual is stored as encrypted data in a verified health pass (Step C).

FIG. 3 shows a schematic representation of how the verified health pass can be used to allow entry of the individual to a venue (Step D) and linked to a proximity detection device (Step E) to allow collecting contact tracing data without the use of a mobile phone inside the venue (Step F).

FIG. 4 shows a schematic representation of how the contact tracing data can be used to identify individuals that have come into contact with an individual being infected with a contagious disease (Step G) and how these potentially infected individuals can be notified and prompted to take the necessary measures to prevent spreading of the disease (Step H).

### Process for COVID-19 Testing (Step A)

As represented in FIG. 1, issuance of a verified health pass may involve obtaining a verified health status that is indicative for the presence or absence of an infection with a disease. COVID-19 (abbreviated as “COVID”) will be used as an exemplary disease in the following, yet it is readily understood that any other disease may be chosen instead. To this end, the individual may book a face to face consultation 10 or online consultation 12, e.g. using a medical booking service.

An online consultation 12 may be conducted with an approved online COVID testing telemedical service 14 that delivers and collects a COVID specific test and is also responsible for safe disposal thereof. The testing shall comply with local health authority regulations using approved POC (point of care) or lab-based tests. For determining a COVID infection status, a combination of antibody IgG/IgM and antigen testing may be used.

The test results 16 are analyzed and the infection status determined by a health care professional 14.→Step A

The infection status is communicated to the tested individual preferably in form of a scannable code via an application software, whereby no detailed medical is information transmitted. The infection status may also be uploaded to a secure server. One example of a server provider is BayWa IT GmbH, Munich, Germany.

The infection status may be issued in form of a green or red status: A green status is issued if no evidence of current infection (through an antigen test) or past infection is found. A green status is also issued if evidence of past infection (e.g. only IgG antibodies), but no evidence of current infection is found (assumed immunity). On the other hand, if evidence of ongoing infection is found, e.g. if IgG and IgM antibodies or antigens for COVID are found, a red infection status is issued.

### Process for ID Verification and VIP Issue (Steps B and C)

Verification of an individual's identity may be done online by comparing a picture or video of the individual's face against a picture of his/her identity card.→Step B

In one embodiment, the verifying of an individual's identity is done with the aid of a video identification service called “WebID” from WEBID SOLUTIONS GMBH. One example of video identification is described e.g. in U.S. Pat. No. 9,911,035. To this end, the individual starts video identification using the services from WebID. A member of the WebID team then guides the individual through the legitimation process, checking the individual's data and ID document. At the end, the individual will be asked to enter a TAN number online. The TAN is sent to the individual via SMS once the identity details have been confirmed.

The individual obtains a verified infection status and provides said infection status to an ID verification service.

The ID service transmits the verified ID and infections status to a secure server 18, preferably from a service provider specialized in secure data transmission and storage. The secure server issues a verified health pass 20 with a QR code 22 encoding for a positive infection status (e.g. indicated in red colour) or negative infective status (e.g. indicated in green colour).→Step C. A negative infection status may be required for gaining entry to a venue.

The verified health pass may also be required to complete a booking e.g. for an event or flight. During the booking process an individual may be asked to scan the code on the individual's verified health pass and the booking can only be completed if e.g. the health status meets certain criteria, such as absence of an infection.

A key aspect of the inventive method is that a verified identity of an individual can be linked to a verified health status of this individual.

Instead of providing information on an individual's identity and/or a verified health status to a remote server of a service provider, said service provider may also provide an ID verification system that uses video streaming technology for both the ID verification and verification of a health status in an online medical consultation. In one embodiment the identity verification process and a medical consultation for obtaining a verified health status (e.g. including entry of medical data, tests completion and entry of results) is done within the same video stream, in which the individual to be tested is identified and is constantly observed or captured on the video stream during the medical consultation. Uninterrupted video streaming ensures that the identified individual cannot be exchanged for the medical consultation.

In another example, an uploading of verified test results and verification of an individual's identity can be done in the presence of a health care professional during a face to face consultation. For instance, an individual may have a real consultation with a health care professional to have a virus test, e.g. an antigen gest and/or an antibody test, conducted for a specific disease. While waiting for the tests results, an online identification of the individual can be conducted in the presence of the health care professional before the latter can upload and verify the test results to the server of the service provider for issuing the verified health pass. Thereby, the identity of the individual linked to specific health data and/or test results is guaranteed.

### Venue Entry and Contact Tracing (Steps D and F)

To allow contact tracing inside a venue 30, the venue is equipped with (GPS independent) high resolution Real Time Locating System (RTLS). For entry, an individual presents the QR code 22 of the individual's verified health pass at venue entry, together with some identification 24, e.g. ID card, driver license or passport. Such an identification document may also be contained in the verified health pass, e.g. included in a specific app running on the individual's mobile phone. A staff member scans the QR code 22 at the entry and confirms photo identity 24 of the individual. Scanning of the QR code triggers generation of a unique identification code and further triggers transmission of time, date and location of the QR scan to a secure server 26→Step D. This data is encrypted before transmission.

The venue issues proximity detection devices 40 in form of tracking bracelets. At the entry, the code on an individual's verified health pass is scanned to generate the mentioned unique identification code and each individual is provided with a proximity tracking bracelet. Through transmission of the unique identification code, the associated tracking bracelet is linked to the verified health pass on the mobile phone or other communication device of the respective individual→Step E. This way, each bracelet is linked to the respective individual, but apart from the unique identification code no other data is exchanged between the tracking bracelet and the smartphone or other personal device on which the verified health pass is stored. Also, the tracing is independent on the use of an individual's mobile phone inside the venue and can therefore also be applied in venues in which mobile phones must be switched off or are not allowed at all. Nonetheless, if desired, tracking data obtained from separate mobile phone based tracking apps may be combined with the data obtained from the tracking bracelet.

All individuals being admitted to the venue may move freely throughout the venue. Using RTLS, the tracking bracelets record interactions between two individuals if an interaction is e.g. within contact distance of less than 2 m and for e.g. at least 5 seconds→Step F. These parameters can be adjusted, depending on the nature of the spreading mechanisms of a specific disease or common diseases. The data on recorded interactions may be stored in the memory of the bracelet and transmitted after the return of the bracelet, or the data may also be transmitted continuously to a secured cloud server in real time.

The recorded data on interactions is only accessed in the event of an individual being later diagnosed with an infectious disease in order to determine all individuals that have come into close proximity of the infected individual.

By making sure that only individuals whose verified health status meets certain criteria, e.g. with respect to an infection or immunity status, the method of the present invention allows for large numbers of individuals coming together in a venue even in case of a pandemic crisis.

The contact tracing preferably occurs using RTLS technology and thus independent of GPS. RTLS allows for highly accurate tracking of the location of a tracking device also inside of a building. Instead of tracking bracelets, any other device that can be attached to an individual—or even implanted—may be used. For the contact tracing itself, no transfer of personal or medical information is required. The only link between the identity of an individual and the contact tracing device is the unique code of the associated contact tracing device that is linked to the mobile phone or other smart device of the respective individual.

### Retesting and Tracing (Steps G and H)

In particular in times of a pandemic crisis due to an outbreak of an infective disease, scheduled retesting is preferably conducted in accordance with local health authority recommendations. For instance, each individual that is not quarantined should be tested in weekly intervals or any time that an individual shows symptoms of an infection. Depending on the outcome of the test, the pertaining health status in the verified health pass is revalidated as “safe” (green pass 46) or changed to “infected” (red pass 48). People identified as infected may further receive recommendations to remain quarantined until their health status is determined as safe.

Apart from green and red, the verified health pass may also have a yellow status indicating that re-validation of a specific health status is required. For instance, if an individual 42 having entered a venue recently is diagnosed with an infectious disease X, every individual that has come into close contact with the infected individual can be identified (→Step G) and their verified health pass of could be changed from green to yellow 47 and prompted to get tested for the infectious disease X→Step H.

The potentially infected individuals 44 may be informed of the change of status of their verified health pass, e.g. by push-notification.

1. A method of issuing a verified health pass to be stored on an individual's mobile device, the method comprising the steps of:
verifying an individual's identity;
receiving at least one verified health status of the individual, said verified health status being indicative of at least one health parameter and being determined by a health care authority;
transmitting the verified health status and verified identity to a remote server to create a verified health pass, said verified health pass including an optical or digital code containing encrypted information on the identity and the at least one health status of the individual; and
transmitting the verified health pass to a mobile device or wearable of the individual.

2. The method as claimed in claim 1, wherein the identity verification is performed by a remote ID verification service and preferably involves video identification or involves uploading of a photo identity of a passport or ID card and a facial photo to the ID verification service.

3. The method as claimed in claim 1, wherein the verified health status includes information of at least one of i) immunity status, ii) infection status, iii) inflammation status and iv) vaccination history.

4. The method as claimed in claim 3, wherein the infection status and/or immunity status is determined based on the presence or absence of antibodies for a specific disease.

5. The method as claimed in claim 1, wherein the verified health status expires after a predefined time limit.

6. The method as claimed in claim 1, wherein the optical code is a QR code or a barcode and preferably only contains encrypted information on the verified health status and identity of the individual.

7. The method as claimed in claim 1, wherein determination of a specific health status includes risk stratification based on predictive analytics.

8. The method as claimed in claim 1, wherein the verified health pass allows entry of the individual to a venue, if a specific verified health status meets at least one predefined criterion, said predefined criterion preferably including at least one of
i) absence of infection,
ii) presence of immunity to a specific disease,
iii) absence of recent intercontinental travel history,
iv) absence of recent travel history into countries presenting a health risk,
v) lasting vaccination for a specific disease, and
vi) absence of a specific health issue.

9. A method for contact tracing in a venue, the method comprising the steps of:
providing a plurality of proximity detection devices each of which including a proximity sensor, a memory, and a battery, and being configured to enable reception, storage and transmission or read-out of data,
for each individual entering a venue, scanning or receiving a code, verifying the individual's identity and generating a unique identification code linked to the individual's identity;
associating one of the proximity-detection devices to each individual and via transmission of the individual's unique identification code to the associated proximity-detection device;
with the aid of the proximity sensor detecting and recording interactions between two or more individuals if an interaction is within the criterion thresholds of least one predefined criterion selected from the group of duration, proximity, and frequency of the interaction; and
transmitting the information on recorded interactions to a secure server database.

10. A method for contact tracing in a venue, the method comprising the steps of:
providing a plurality of proximity detection devices each of which including a proximity sensor, a memory, and a battery, and being configured to enable reception, storage and transmission or read-out of data,
for each individual entering a venue, scanning or receiving an optical or digital code on a verified health pass,
the verified health pass being stored on a mobile device and the optical or digital code containing encrypted information on the identity and at least one health status of the individual; said at least one health status being indicative of at least one health parameter and determined by a health care authority;
checking if the at least one health status meets predefined criteria for allowing entry to the venue;
verifying the individual's identity and generating a unique identification code linked to the individual's identity;
associating one of the proximity-detection devices to each individual and via transmission of the individual's unique identification code to the associated proximity-detection device;
with the aid of the proximity sensor detecting and recording interactions between two or more individuals if an interaction is within the criterion thresholds of least one predefined criterion selected from the group of duration, proximity, and frequency of the interaction; and
transmitting the information on recorded interactions to a secure server database.

11. The method as claimed in claim 9, wherein the tracking occurs in a venue equipped with Real Time Locating System (RTLS).

12. The method as claimed in claim 9, wherein the predefined criterion and/or the criterion threshold is adjustable by a program stored in the proximity detection device.

13. The method as claimed in claim 9, wherein an interaction is recorded if two proximity detection devices come into contact at a distance of less than 1.5 m, preferably less than 2 m.

14. The method as claimed in claim 9, wherein the proximity detection device is sized and configured to be worn around a body part of the individual.

15. The method as claimed in claim 9, wherein the proximity detection device is securely attached to the associated individual upon entering a venue and detachment of the device preferably requires the aid of an authorized individual.

16. The method as claimed in claim 9, wherein scanning of the optical code triggers transmission of time, date and location data to the secure server database.

17. The method as claimed in claim 9, wherein, in case of an individual being diagnosed with an infectious disease, the method further includes the steps of:
accessing the database and based on the recorded interactions by the proximity detection device associated to the individual carrying the disease, and
determining potentially infected individuals.

18. The method as claimed in claim 17, wherein the potentially infected individuals are informed, preferably by sending a notification to their mobile devices.

19. The method as claimed in claim 9, wherein the verified health status on the mobile device of potentially infected individuals is changed.
